(19) United States
(12) Patent Application Publication
(10) Pub. No.: US 2019/0282380 A1
(43) Pub. Date: Sep. 19, 2019

(54) ACOUSTIC MUFFLER SOCK FOR PROSTHETIC DEVICE LINER OR PROSTHETIC DEVICE

(71) Applicant: Bonnie S. Schnitta, East Hampton, NY (US)

(72) Inventor: Bonnie S. Schnitta, East Hampton, NY (US)

(21) Appl. No.: 16/299,183

(22) Filed: Mar. 12, 2019

Related U.S. Application Data
(60) Provisional application No. 62/642,046, filed on Mar. 13, 2018.

(51) Int. Cl. A61F 2/78

(52) U.S. Cl. CPC A61F 2/7812; A61F 2002/5087; A61F 2002/7818

(57) ABSTRACT

An acoustic muffler sock for use with a prosthetic device liner or with a socket of a prosthetic device includes a first sock opening at one end and sound absorbing material designed to inhibit noises that are often created from the liner/amputated limb interaction. The acoustic muffler sock is configured to be positioned over the liner before the liner and the acoustic muffler sock thereon is inserted into the socket for the prosthetic device. The acoustic muffler sock may further comprise a sound barrier material. The muffler can also be part of a sock that goes over the prosthetic device.

### CROSS-REFERENCE TO RELATED APPLICATIONS

This application derives the benefit of the filing date of U.S. Provisional Patent Application No. 62/642,046, filed Mar. 13, 2018, the content of which provisional application is incorporated herein by reference.

### BACKGROUND OF THE INVENTION

The present invention relates broadly to prosthetic device liners and, more particularly relates to a prosthetic limb or liner cover, sock or sock cuff configured to act as an acoustic muffler (i.e., an “acoustic muffler sock”) to inhibit sounds that often exit a prosthetic liner at the top and on occasion, through the prosthetic liner or the prosthetic device itself.

Liners for a prosthetic limb are used to cushion transfer loads and reduce friction from the prosthetic components to protect the skin between the prosthetic socket and the residual limb. Also, liners provide additional functionality by contributing to the suspension of the prosthesis.

Suspension usually relies on the tenacity with which the liner maintains its grip on the residual limb. Satisfactory performance of the prosthesis as a whole depends on many factors, but the quality of the fit or grip of the liner on the residual limb is essential. Unfortunately, this grip, due to the poor heat transfer of the liner and often also the prosthesis, increases the temperature in the liner, causing sweating and moisture buildup.

This collection of moisture leads to the development of an audible “burping or squeaking noise.” In addition to the complete nuisance of this noise, often transmitted with every step, the prosthetic wearer cannot be quiet in their approach and often feels embarrassed and sometimes even ashamed preventing them from participating in activities, or avoiding attendance of certain social settings. This acoustic burden can have a drastic negative impact on quality of life along with the patient's mental health, especially to the most vulnerable amputees who are already having a difficult time coping with limb-loss-grieving, depression, PTSD, low self-esteem and in many instances leading to social isolation. In addition, this increase in sweating leads to a poorly fitting socket and in some patients the prosthesis falling completely off the residual limb. The medical sequela include dermatitis/skin irritation/sores/ulceration, Verrucous hyperplasia, skin breakdown/sensitivity, skin infection, increase in stump pain, exacerbation of nerve pain, and increase in phantom sensations. The only treatment is prolonged disuse of the entire prosthesis leading to the inability of the amputee to ambulate for many weeks at time.

Historically, liners have been made from open and closed cell foams formed around the residual limb. Foam liners are still used in a few clinics today but the overwhelming majority of modern liners are primarily made of an outer fabric layer and an inner elastomeric layer, comprising silicone, a thermoplastic elastomer, or polyurethane; such foam liners are essentially “rolled-on” to the residual limb, in practice. These more modern rolled liners are believed to offer better suspension, durability, and cushioning than foam.

The primary reasons liners or socks have failed/or are not worn by patients, have to do with comfort, providing the proper cushion (i.e. protect skin integrity) & the feel of the material against the skin. If the material is not comfortable or causes skin irritation, rash, etc., no amputee will wear the prosthesis no matter how well it performs heat transfer/dissipation. These are the main reasons why silicone and gel are found in the majority of liners.

Regardless of the choice of the liner material, method of suspension, etc., there nevertheless is the “squeak and burp” noise issue, as explained above, and other noises that occur that reduces the amputee's quality of life. The inventive acoustic muffler sock works with all methods of suspension and methods of donning and doffing of the prosthesis to resolve this critical noise issue for amputees. Although this acoustic muffler sock solves this horrible noise problem for amputees, there are other applications for the inventive acoustic muffler sock, as now explained in further detail.

### SUMMARY OF THE INVENTION

The present invention overcomes the shortcomings of known arts, such as those mentioned above. For example, and in an embodiment, the invention provides an innovative prosthetic limb cover, sock or sock cuff, or prosthetic liner, sock or sock cuff, engineered to act as an acoustic muffler (i.e., an “acoustic muffler sock”) to inhibit the sounds that often exit the prosthetic liner at the top and on occasion, through the prosthetic limb cover, liner, sock, sock cuff or the prosthesis itself.

The inventive acoustic muffler sock can be a full sock that replaces the standard sock that amputees wear, with a muffler top that tucks into or over the liner or remains above the liner, or the acoustic muffler sock can be just the muffler itself that is attached at the top of the liner, prosthetic, another sock, etc., without limitation, if configured according to the inventive principles set forth below herein. The inventive acoustic muffler sock is either tucked into the liner or remains above the liner. The inventive acoustic muffler sock with muffler can either be just a sock, an acoustically absorptive sock, or an acoustic absorptive/barrier sock that can absorb and stop the sounds that often exit out of the liner at the top, or from the liner, or from the liner and prosthesis before the sounds reach and exit from the top of the liner. The muffler is made with a sound absorbing layer that may be any type of material that can be made into a wearable attachment to the prosthetic limb or liner, including but not limited to weaves, knits, neoprene with an absorbent layer, organic or inorganic material, antibacterial or microperforated, etc.

### BRIEF DESCRIPTION OF THE DRAWING FIGURES

FIG. 1 depicts an embodiment of an inventive acoustic muffler sock of the invention;

FIG. 2 depicts a liner for a residual limb, into which a top of the inventive acoustic muffler sock is positioned or tucked into for intended use;

FIG. 3A depicts another embodiment of the inventive acoustic muffler sock of the invention;

FIG. 3B depicts a cross-section of a material from which the embodiment of the acoustic muffler sock of FIG. 3A is formed;

FIG. 4 depicts another embodiment of an acoustic muffler sock of the invention;

FIG. 5 depicts another embodiment of an acoustic muffler sock of the invention;

FIG. 6 depicts a liner to which an acoustic muffler sock, such as depicted in FIG. 4 or 5 is attached; and

FIG. 7 depicts a prosthetic device configured according to the invention.

### DETAILED DESCRIPTION OF THE INVENTION

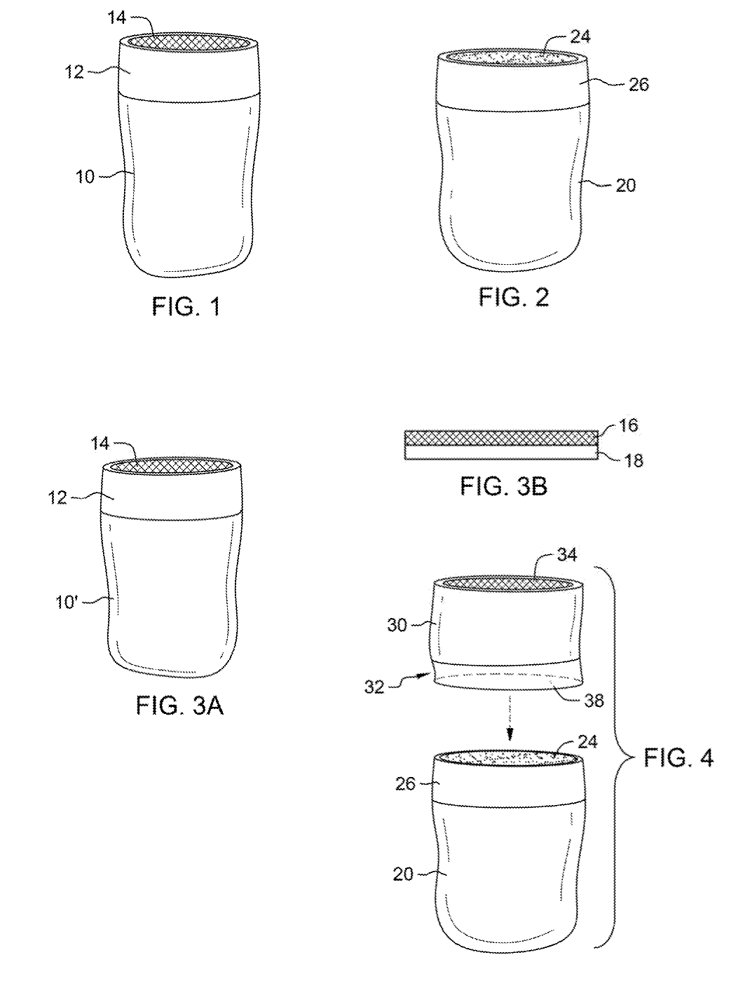

A first embodiment of the inventive acoustic muffler sock is depicted in FIG. 1. As shown therein, the acoustic muffler sock 10 is an entire sock that is comprised of sound absorbing material. The sound absorbing material may be any type of material or combination of materials that absorbs sound energy. The top 12 of the acoustic muffler sock 10, positioned proximate sock opening 14 is either tucked into a liner for a residual limb, such as a liner 20 shown in FIG. 2, with an attachment segment 26 or extends above and beyond the upper or open end 24 of the liner 20. In the embodiment shown, the acoustic muffler sock 10 preferably is worn over the liner 20, or over the prosthesis.

In another embodiment, an acoustic muffler sock 10′ is depicted in FIG. 3A. As shown therein, the entire sock is comprised of sound absorbing and barrier material. FIG. 3B depicts an embodiment of the material from which the material from which the sock 10′ of FIG. 3A is formed. That is, the sock 10′ comprises sound absorbing material 16 and a sound barrier material 18. The sound absorbing material 16 may be any type of material or combination of materials that absorbs sound energy. The barrier material 18 may be something as simple as one thin layer or more of Lumitex distributed by SoundSense, or any sound barrier product with the required transmission loss in the frequencies of disturbance to inhibit the disturbing noise from exiting through the liner and prosthesis prior to that sound reaching the top of the acoustic muffler sock.

The barrier material 18 may comprise a moldable loaded vinyl, or like barrier material. Preferably, the layer of sound barrier material 18 is configured as an outer layer, but the invention is not limited thereto. Moreover, the invention may comprise two layers of barrier material surrounding a layer of sound absorbing material or two layers of sound absorbing material surround a layer of sound barrier material. The top of the acoustic muffler sock 10′ is either tucked into a liner for a residual limb, or extends above and beyond the liner. In the embodiment shown, the acoustic muffler sock 10′ preferably is worn over the liner 20.

In another embodiment, an acoustic muffler sock 30 is depicted in FIG. 4. The acoustic muffler sock 30 includes an attachment segment 32 proximate lower opening 38 but includes a second opening at the muffler top. The muffler 30 is attached to the top of a liner 20, also depicted in FIG. 4. For that matter, acoustic muffler sock 30 may be attached directly to a sock, a prosthesis, a liner, or prosthesis socket. This acoustic muffler sock embodiment 30 is comprised of sound absorbing material that may be any type of material or combination of materials that absorbs sound energy; A bottom portion 32 of this acoustic muffler sock 30 is tucked into the liner or is attached to attachment segment 26 and extends above the liner. In the embodiment shown, the acoustic muffler sock 30 preferably is worn above the liner 20.

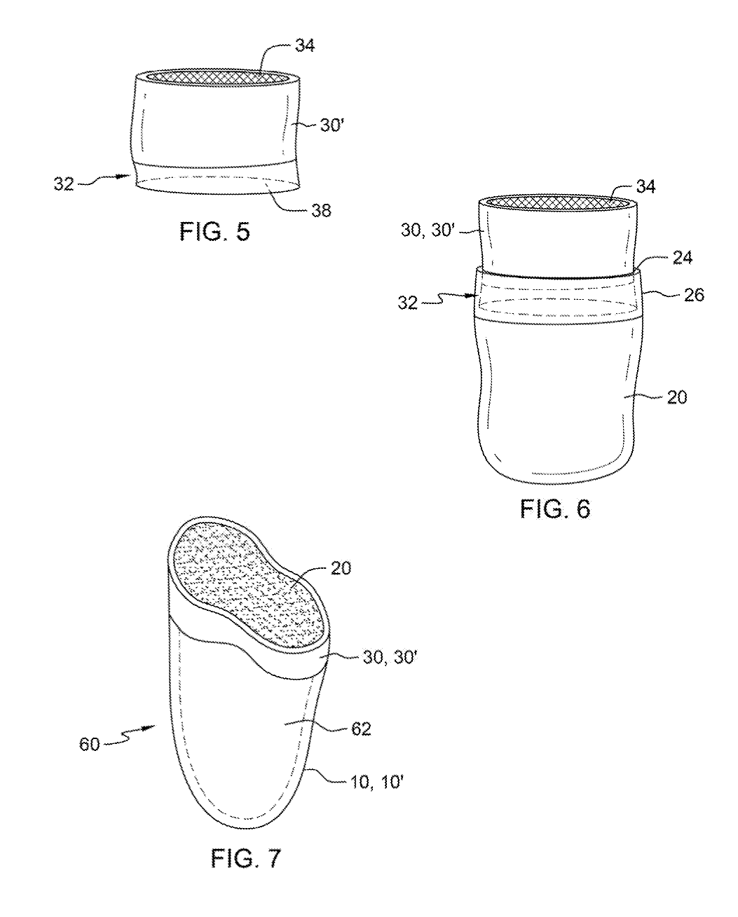

In another embodiment, an acoustic muffler sock 30′ is depicted in FIG. 5. Like the acoustic muffler sock 30 (FIG. 4), the acoustic muffler sock 30′ includes an attachment segment 32 proximate lower opening 38 and includes a second opening at the muffler top. The acoustic muffler sock 30′ is configured to be attached to the top of a liner 20, a sock (not shown), a prosthesis, or a prosthesis socket (see FIG. 7). This acoustic muffler sock 30′ is comprised of sound absorbing material 16 and barrier material 18. The sound absorbing material 16 may be any type of material or combination of materials that absorbs sound energy.

The barrier may be something as simple as one thin layer or more of Lumitex distributed by SoundSense, or any product with the required transmission loss in the frequencies of disturbance to inhibit the disturbing noise from exiting through the liner and prosthesis prior to that sound reaching the top of the acoustic muffler sock, such as a moldable loaded vinyl, without limitation. The top of acoustic muffler sock is tucked into the liner or is attached to the attachment segment 26 and extends above the liner. In the embodiment shown, the acoustic muffler sock 30′ preferably is worn above the liner 20.

FIG. 6 shows a liner 20 to which a muffler sock 30 or 30′ is attached at the top where the top portion 32 is not folded in. The sound absorbing acoustic muffler sock 10, 10′ and acoustic muffler sock 30, 30′ are adhered to or are otherwise arranged to be integral with at least a part of a surface of the liner 20 or the prosthesis to which a user's limb portion is to be attached. The means to effect such adherence or connection may be any means known to a person of ordinary skill in the art for adhering, attaching, sewing, or applying one material (for example, the sound absorbing material 16 or the sound barrier material 18, or both where the two materials are formed together as a blend (not shown)), to a surface or a portion of a surface a sheet of material comprising cloth or polymer, from which the liner 20 is typically formed.

The resulting acoustic muffler sock 10, 10′ or acoustic muffler sock 30, 30′, with the sound absorbing 16 material or sound absorbing 16/barrier layer 18 material, are preferably flexible so that they may be configured to function as a boundary to unwanted sound, and stay close and preferably tight to the skin and/or liner of the user, so that the sound generated in the confine of the liner or prosthetic socket or device is forced to travel thru the muffler before exiting the sides, top or opening. This is inclusive of allowing the sock to be of any color or design that the amputee feels best suits their liking.

In another embodiment, as shown in FIG. 7, the invention provides a prosthetic device 60, including a socket portion 62, for attaching to a remainder portion of a limb (not shown) from which another portion has been amputated. The inventive prosthetic device 60 comprises, among other elements, a liner 20 and an acoustic muffler sock 10, 10′, 30, 30′ for attachment directly to the liner 20, or the prosthetic device 60 (the socket 62 in particular) or both. As explained above, the acoustic muffler sock comprises sound absorbing material 16, and may further comprise a sound barrier material. The acoustic muffler sock is preferably formed with one layer of the sound absorbing material and one layer of sound barrier material, with the barrier layer being an outer layer. However, the barrier layer may be sandwiched between two sound absorbing layers. For particular applications the sock may be formed with multiple layers or sound absorbing and sound barrier materials. For that matter, and as explained above, the sock may be formed as a blend of sound absorbing and sound barrier material. The blend preferably comprises a range of 40 to 95 weight percent sound absorbing material and 5 to 60 percent of sound barrier material, but preferably 85% and 15% by weight of sound absorbing and sound barrier material, respectively. As shown, the acoustic muffler sock is configured to be positioned over the liner 20 before the liner 20 and the acoustic muffler sock 10, 10′, 30, 30′ thereon is/are inserted into the socket portion 62 of the prosthetic device.

As will be evident to persons skilled in the art, the foregoing detailed description, applications and figures are presented as examples of the invention, and that variations are contemplated that do not depart from the fair scope of the teachings and descriptions set forth in this disclosure.

What is claimed is:

1. An acoustic muffler sock for use with a prosthetic device liner or with a socket of a prosthetic device, the acoustic muffler sock comprising:
a first sock opening at one end; and
sound absorbing material;
wherein the acoustic muffler sock is configured to be positioned over the prosthetic device and the acoustic muffler sock thereon is inserted into or above the liner for the prosthetic device.

2. The acoustic muffler sock as defined by claim 1, further comprising a sound barrier material.

3. The acoustic muffler sock as defined by claim 2, wherein the sound absorbing material and the sound barrier material are formed as separate layers, and wherein the layers are attached to or integral with each other.

4. The acoustic muffler sock as defined by claim 2, wherein the sound absorbing material and the sound barrier material are formed as a blend.

5. The acoustic muffler sock as defined by claim 3, wherein during use, the sound absorbing material layer contacts the prosthetic device and the sound barrier layer faces out from the prosthetic device.

6. The acoustic muffler sock as defined by claim 1, wherein during use, a portion of the sock extends beyond an opening end of the prosthetic device.

7. The acoustic muffler sock as defined by claim 6, wherein during use, the portion of the sock extends beyond an opening end of the prosthetic device and is turned over and tucked into the liner, such that the portion is positioned between an inner liner surface and a user's limb part for attachment.

8. The acoustic muffler sock defined by claim 1, further comprising a second opening on an end of the sock that opposes the first sock opening.

9. A liner for a prosthetic device, comprising:
an acoustic muffler sock comprising:
a first sock opening at one end; and
sound absorbing material;
wherein the acoustic muffler sock is configured to be positioned over the prosthetic device before the liner is inserted into the socket for the prosthetic device.

10. An acoustic muffler sock for use with a prosthetic device liner or with a socket of a prosthetic device, the acoustic muffler sock comprising:
a first sock opening at one end; and
sound absorbing material;
wherein the acoustic muffler sock is configured to be positioned over the liner before the liner and the acoustic muffler sock thereon is inserted into the socket for the prosthetic device.

11. The acoustic muffler sock as defined by claim 10, further comprising a sound barrier material.

12. The acoustic muffler sock as defined by claim 11, wherein the sound absorbing material and the sound barrier material are formed respectively as a first layer of sound absorbing material and a first layer of sound absorbing material, and wherein the layers are attached to or integral with each other.

13. The acoustic muffler sock as defined by claim 11, wherein the sound absorbing material and the sound barrier material are formed as a blend.

14. The acoustic muffler sock as defined by claim 12, wherein during use, the sound absorbing material layer contacts the liner and the sound barrier layer contacts some inner part of the socket of the prosthetic device.

15. The acoustic muffler sock as defined by claim 10, wherein during use, a portion of the sock extends beyond an opening end of the liner.

16. The acoustic muffler sock as defined by claim 15, wherein during use, the portion of the sock extending beyond the opening end of the liner is configured to be turned over and tucked into the liner, such that the portion is positioned between an inner liner surface and a user's limb part for attachment.

17. The acoustic muffler sock as defined by claim 10, further comprising a second opening on an end of the sock that opposes the first sock opening.

18. The acoustic muffler sock as defined by claim 12, further comprising a second layer of sound barrier material and wherein the first and second layer of sound barrier material surrounds the first layer of sound absorbing material.

19. The acoustic muffler sock as defined by claim 12, further comprising a second layer of sound absorbing material and wherein the first and second layer of sound absorbing material surrounds the first layer of sound barrier material.
